(19) United States
(12) Patent Application Publication
(10) Pub. No.: US 2004/0042583 A1
(43) Pub. Date: Mar. 4, 2004

(54) Patient positioning system for radiotherapy/radiosurgery based on stereoscopic X-ray unit

(76) Inventors: Andreas Wackerle, Schrobenhausen (DE); Cornel Schlossbauer, Munchen (DE); Stephan Erbel, Munchen (DE); Stephan Frohlich, Aschheim (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 10/464,079

(22) Filed: Jun. 18, 2003

(30) Foreign Application Priority Data
Jul. 12, 2002 (DE) 102 31 630.9
Jun. 18, 2003 (EP) 03 013 392.0

(51) Int. Cl. A61N 5/10

(52) U.S. Cl. 378/65

(57) ABSTRACT

The invention relates to: a patient positioning system for radiosurgery and/or radiotherapy, in particular with the aid of a LINAC and based on stereoscopic x-ray images; a calibration system, in particular for a patient positioning system; an image recorder and/or detector arranging device, in particular to be used with a patent positioning; and a collision protection means, in particular to be used with a patient positioning system or an image recorder and/or detector arranging device.

The invention relates to a patient positioning system for radiotherapy/radiosurgery, based on a stereoscopic x-ray apparatus. To improve radiosurgery/radiotherapy, accurate positioning of the target region is necessary. Stereoscopic x-ray imaging, and combining with DRRs, represent a way of exactly positioning patients for radiotherapy. In this case, the following marginal parameters must be taken into account:

the table and gantry rotation of the LINAC must not be restricted, and/or the x-ray apparatus must under no circumstances collide with the linear accelerator or the patient;

the x-ray detectors must under no circumstances be exposed to the “hard” radiation of the LINAC;

the target region of the LINAC (the area around the target point) is to be imaged in both images. The images can be taken in the treatment position.

precision is at its highest when the apparatus manages without mechanically moving parts (or with as few as possible);

the apparatus is to be ready for operation at any time, without additional setting-up time. Unfolding and folding back components is therefore undesirable;

access to the patient must not be restricted;

no part is to be attached lower than 1.90 m, in order to avoid “head banging”;

soft kV(40-300 kV) radiation is to be used, for optimum image quality;

the distance between the object (the patient) and the detector is to be as small as possible.

A patient positioning system in accordance with the invention for radiosurgery and/or radiotherapy, in particular with the aid of a LINAC and based on stereoscopic x-ray images, comprises the features of claim 1. The sub-claims define preferred embodiments of the invention. The system advantageously comprises one or more of the following features:

positioning is based on comparing x-ray images shot using the system and DRRs;

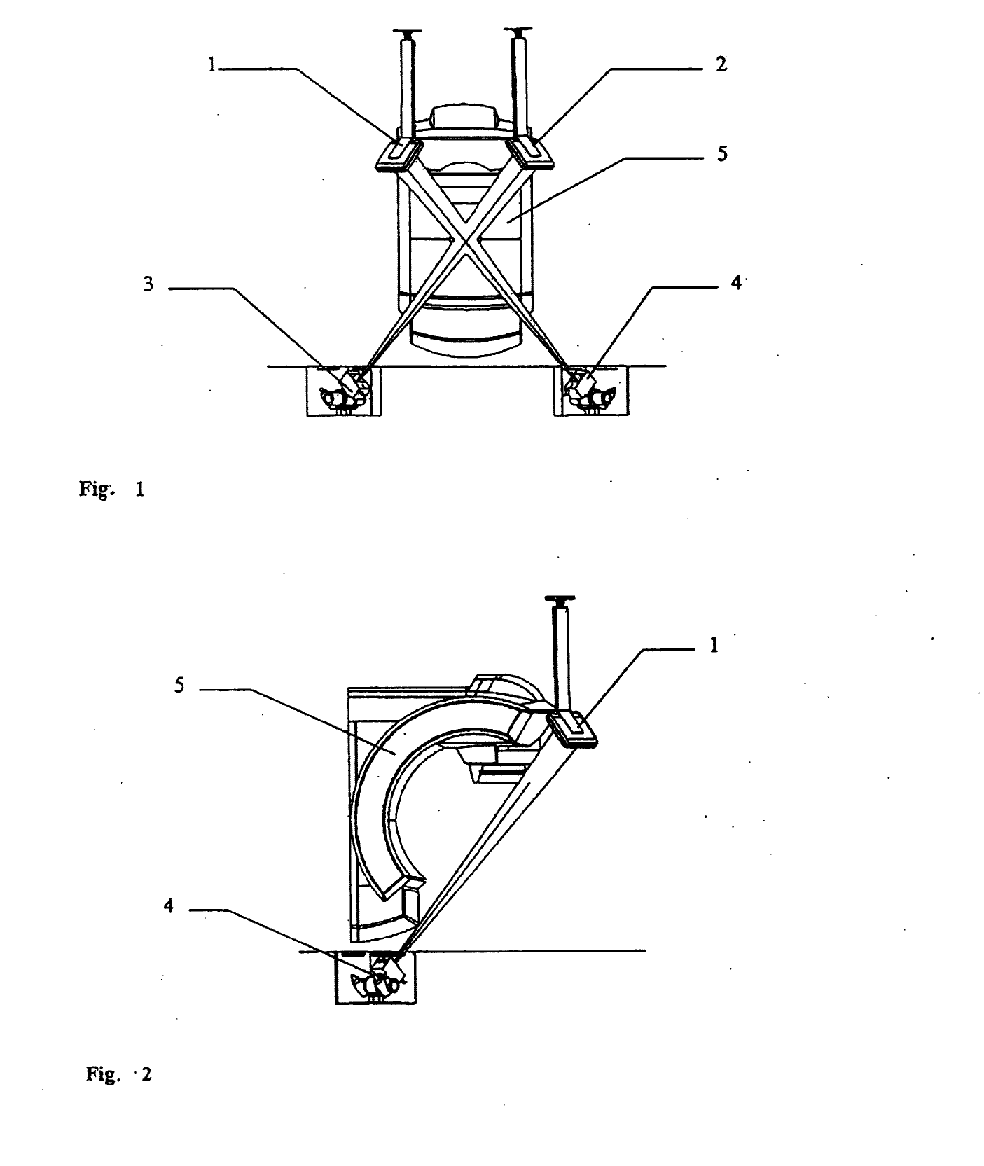

two kV x-ray sources are permanently let into the floor, left and right of the LINAC gantry (see FIG. 1);

two x-ray image recorders (see FIG. 1; for example, a-Si detectors or conventional x-ray image intensifiers) are fixed to the ceiling in front of the gantry, to the left and right above the patient;

the radiation path from the x-ray sources to the image recorders passes through the target area of the linear accelerator (around the isocentre);

any possibility of collision between the LINAC and the x-ray apparatus is ruled out, in particular the gantry and the table can be rotated freely;

the radiation is to be administered in a number of doses/radiotherapy sessions until the target is necrotic, in particular in two, three, four, five or more sessions;

the x-ray images are taken sequentially, spaced out in time, in particular using one generator for both x-ray tubes, which is toggled.

A calibration serves to exactly determine the position (X, Y, Z+ angle) of the x-ray tubes and x-ray detectors spatially with respect to a fixed point fixed in space. This is necessary since the mechanical design can only be precise in the range of a few centimetres, which is not sufficient for the system to function correctly.

The “fixed point fixed in space” can be situated at any point in space, and thus also in the isocentre of the linear accelerator.

If a fixed point fixed in space and lying outside the isocentre is used, then two approaches are in principle possible:

a) The positioning system is used to create a relationship between the position of the target region and one or more “external parameters” or to adapt an already extant relationship. “External parameters” means for example the position of infrared body markers. The target region is then finally positioning, exclusively based on the “external parameters” and their relationship to the target region.

b) The fixed point fixed in space has a defined relationship (offset) to the isocentre. In this case, the offset is simply added to the ascertained positioning error.

In order to explain the elements of the invention is simply as possible, it is assumed in the following that the “fixed point fixed in space” is situated in the isocentre of the linear accelerator.

In order to perform the calibration, it is necessary to know where the x-ray markers used were situated relative to the fixed point fixed in space during stereoscopic x-ray imaging, and where each of their “shadows” is projected on the detector.

The calibration can be performed, within the framework of the embodiment of the present invention, using one or more of the following steps:

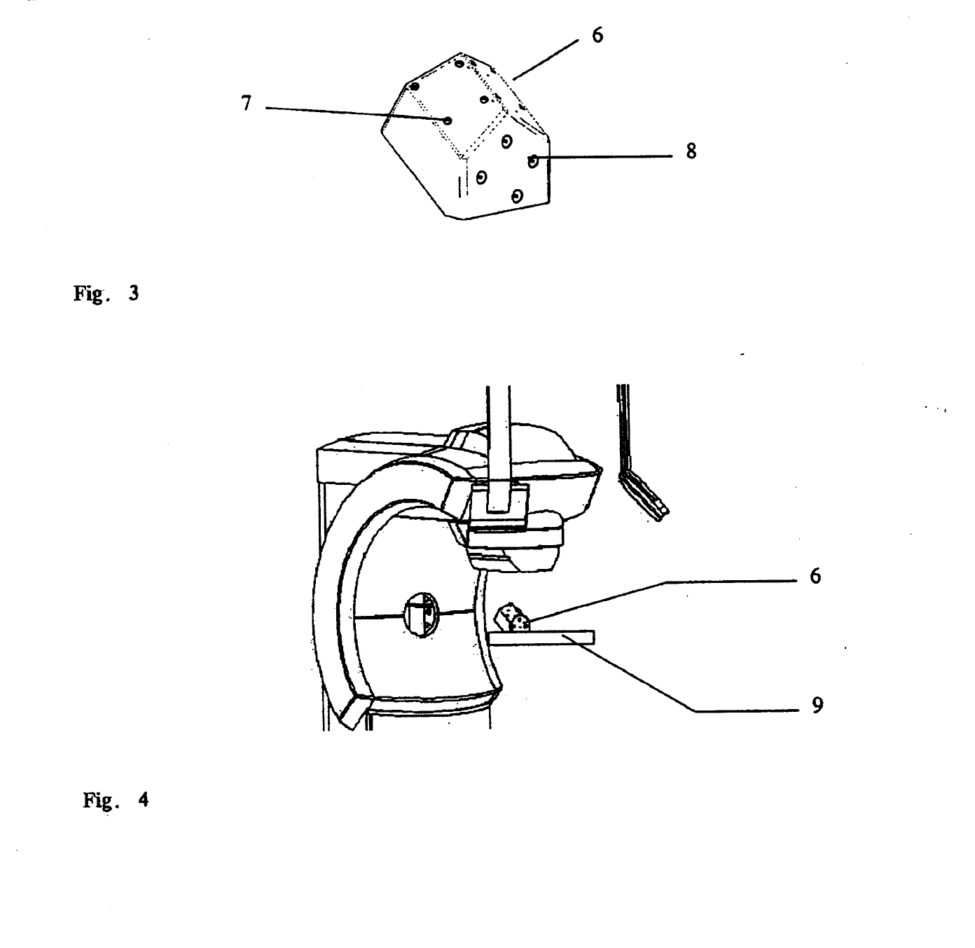

for calibrating, a “phantom” (FIG. 3) is used which is provided with x-ray-visible markings (x-ray markers) which are clearly visible in the x-ray image;

said phantom is additionally fitted with other markers which are detected by an independent 3D measuring system (for example, an IR tracking system);

the x-ray markers and the additional markers have a known, spatially fixed relationship to each other;

the spatial position of the individual x-ray markers is calculated using the position of the additional markers in relation to the fixed point fixed in space, detected by the 3D measuring system;

the spatial position (X, Y, Z+ angle) of the x-ray sources and the x-ray image recorders relative to the fixed point fixed in space is calculated from the spatial position of the individual x-ray markers and their projections onto the x-ray image recorders;

the x-ray system is calibrated, independent of the calibration/referencing of the LINAC system.

The image quality is particularly dependent on the object(patient)-detector distance. The distance features in the formula squared. This also of course applies to the source-object distance; this, however, can be better compensated for by a higher output of the source.

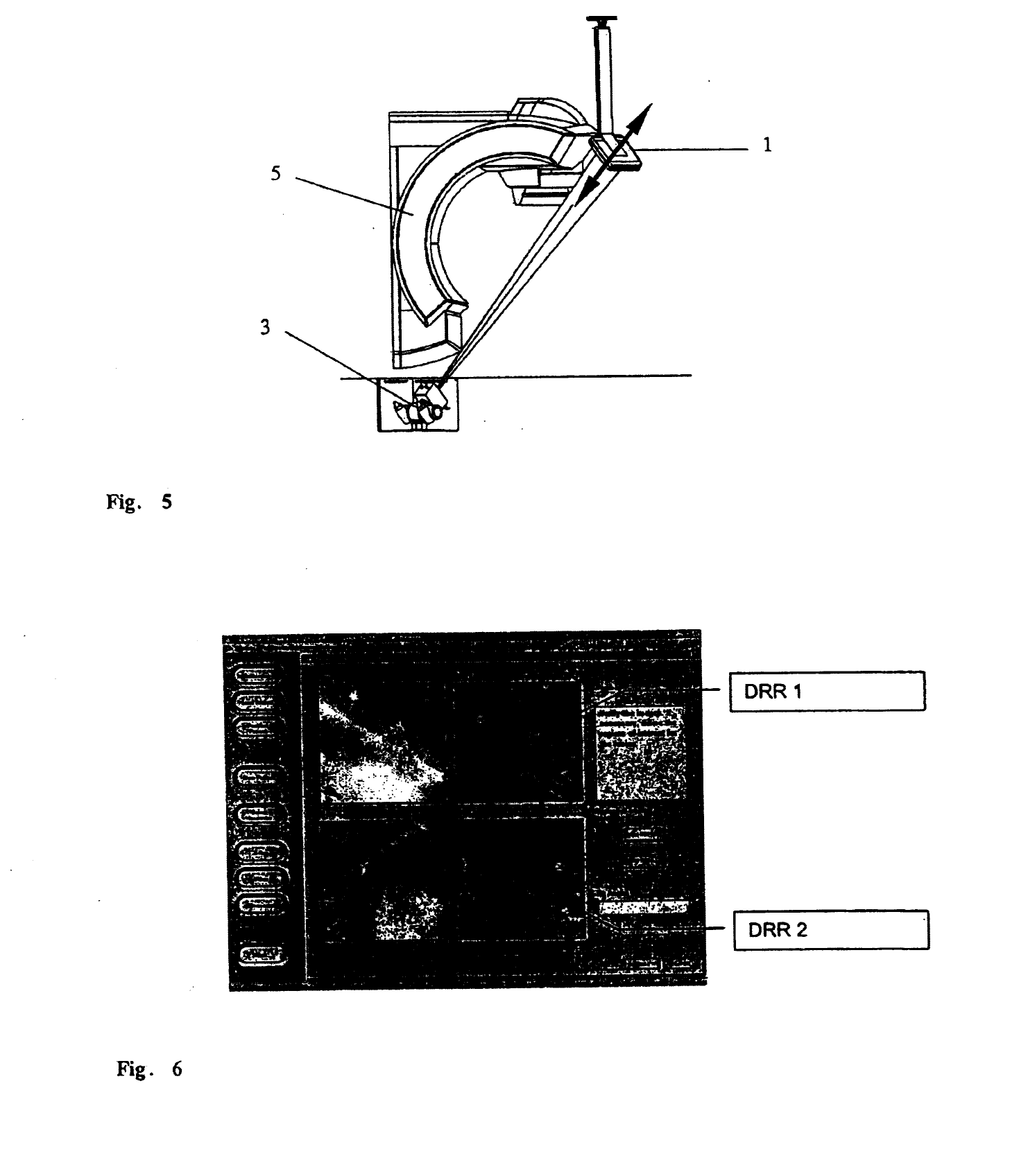

It is therefore advantageous for the detector to be situated as near to the patient as possible. A moving detector is therefore provided (FIG. 5) in accordance with an aspect of the invention.

A patient positioning system in accordance with the invention for radiosurgery and/or radiotherapy, in particular with the aid of a LINAC and based on stereoscopic x-ray images, in accordance with this embodiment, comprises one or more of the following features:

said image recorder (detector) can be moved along the beam direction of the respective x-ray source;

the image recorder is moved manually or using motors;

for taking x-ray images, the image recorder is situated in a position near the patient;

when it is not required, the image recorder is situated in a position away from the patient;

in the position away from the patient, the image recorders do not obstruct the rotation of the gantry and the table.

Among other things, the system in accordance with the invention provides protection against collision, wherein:

the image recorders are equipped with a collision protection system (for example, contact sensors, ultrasound telemeters, laser scanning) and/or

the collision protection system enables the image recorders to be moved automatically (even without an operator).

All the features described herein and in the appended patent claims, individually and in any combination, can be regarded as the subject of the invention. The invention is described in the following by way of an example embodiment and with the aid of the enclosed figures which show individual components and units of the system in accordance with the invention. There is shown:

FIG. 1 a system in accordance with the invention, in a front view;

FIG. 2 a lateral view of the system;

FIG. 3 a calibration phantom;

FIG. 4 a LINAC system comprising a calibration phantom;

FIG. 5 a system comprising a moving image recorder/detector;

FIG. 6 a screen shot for calculating the DRRs;

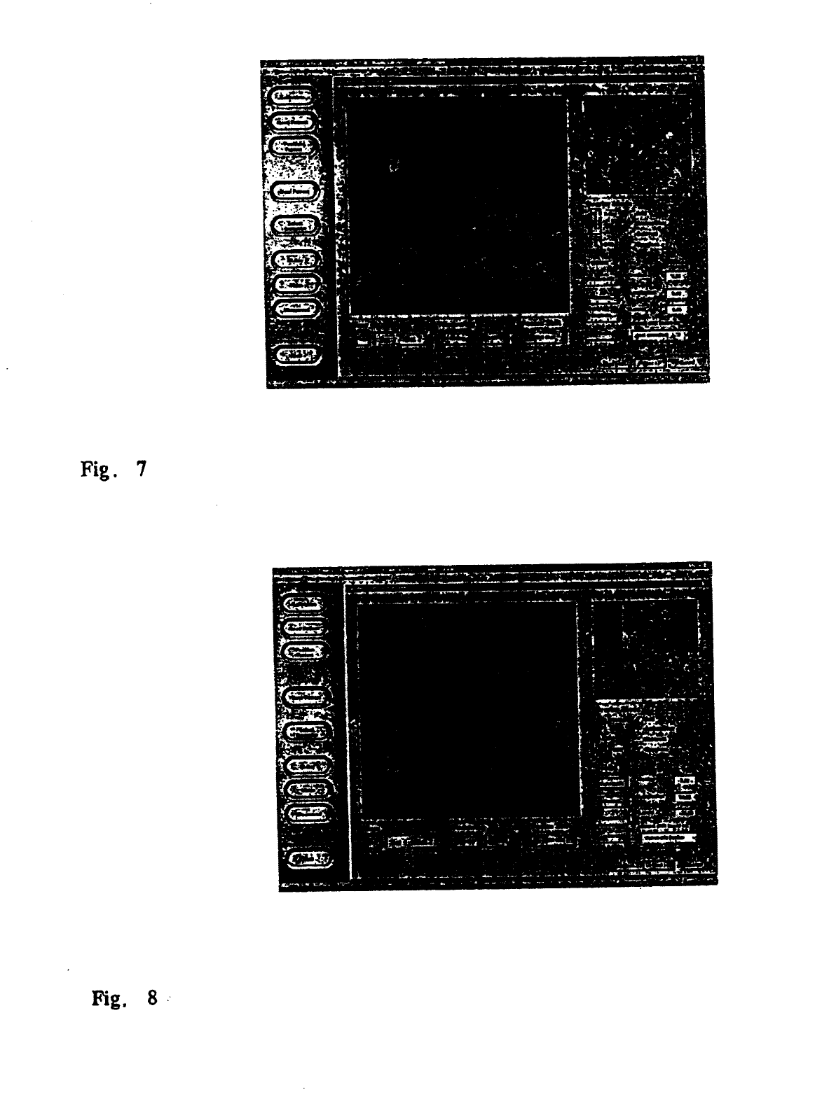

FIG. 7 a screen shot for superimposing the DRRs and the x-ray images;

FIG. 8 a screen shot with the DRRs and the x-ray images overlapped;

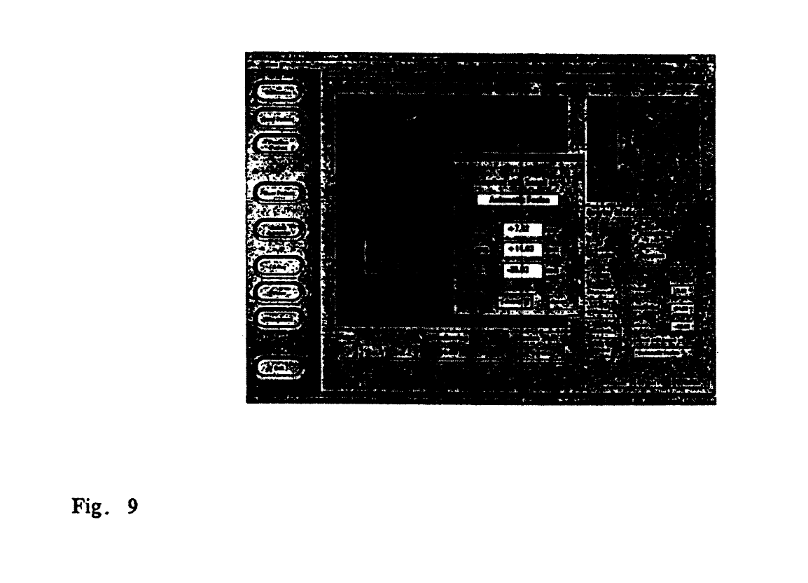

FIG. 9 a screen shot with the necessary correcting shift indicated.

The system shown in FIG. 1 comprises two conventional diagnostic x-ray tubes 3, 4 let into the floor left and right of the linear accelerator gantry 5, and two x-ray detectors 1, 2 fixed to the ceiling. In this example, detectors 1, 2 made of amorphous silicon are used. The angle between the two tube-detector combinations is somewhat less than 90°. FIG. 2 shows a lateral view of the system.

The calibration serves to exactly determine the position (X, Y, Z + angle) of is the x-ray tubes 3, 4 and the x-ray detectors 1, 2 spatially relative to the fixed point fixed in space. This is necessary since the mechanical design can only be precise in the range of a few centimetres, which is not sufficient for the system to function correctly.

For calibration, a “plastic phantom” 6 shown in FIG. 3 is used which is provided both with infrared markers 8, and can therefore be detected by the 3D tracking system, and with x-ray markers 7 (rings similar to washers) which are clearly visible in the x-ray image. Said phantom 6 is positioned near the fixed point fixed in space.

For the actual calibration, two x-ray images (one image per x-ray tube—x-ray detector combination) are taken. The spatial position of the phantom 6 is automatically detected via an IR tracking system (ExacTrac), see also FIG. 4. The position of the image of the x-ray markers 7 in the two x-ray images is likewise automatically detected. With the aid of this information, the system can calculate the spatial position of the tubes 3, 4 and the detectors 1, 2.

The treatment is for example executed as follows:

Pre-Positioning

Advantageously, the patient should already be precisely placed at approx. ±3 cm with respect to the fixed point fixed in space. The patient can be pre-positioned either using a patient marker tracking system or conventionally on the basis of skin markings which are aligned with the 3D lasers. For pre-positioning using the patient marker tracking system, infrared body markers are used. The tracking and positioning system can for example be an ExacTrac system of the firm BrainLAB, Heimstetten, Germany. It will also be called so in the following, although other tracking and positioning systems can in principle also be used.

Conventional pre-positioning is performed without infrared body markers. It may be noted that the subsequent procedure would in principle also work without any markers. Since, however, the movement of the table can be controlled via ExacTrac, some IR markers are provided on the table in this case.

Taking the X-ray Images Two x-ray images (one image per x-ray tube—x-ray detector combination) are then taken. The necessary toggling between the two tubes 3, 4 is performed manually. In accordance with one embodiment, the system does not take the two x-ray images simultaneously but sequentially. The possibility then exists of using a single generator for both x-ray tubes 3, 4, which saves on investment costs. Since the system is capable of tracking the patient and referencing at any point in time, the expense of providing two generators, in order to produce simultaneous x-ray images, is not necessarily required.

Furthermore, if the images are produced sequentially, this can ensure that the scattered radiation of one x-ray image does not interfere with the image on the other detector 1, 2. The detectors 1, 2 can then advantageously be moved very near to the target point and also very near to each other, as for example shown in FIG. 5; scattering filters can be omitted.

Calculating the DRRs (digital reconstructed radiographs)

Two DRRs are then calculated which indicate the “nominal image content”. The screen shot in FIG. 6 shows an example.

For this, the system uses the following information: the spatial position of the tubes 3, 4; the position of the detectors 1, 2; the position of the fixed point fixed in space; the 3D CT data set, and the position of the planned target point relative to the 3D CT data set (=planned isocentre) from the planning system.

If the patient is already lying absolutely correctly, then the DRRs will match the actual x-ray images 100%, aside from any brightness settings and images of the patient bed. If the patient is not correctly placed, then the DRR-x-ray image pairs will exhibit differences in the image detail.

Superimposing and Fusing the DRRs and the X-Ray Images

The DRRs and the x-ray images are superimposed on the screen, as for example shown in the screen shot in FIG. 7.

If necessary, the x-ray images are then shifted such that either the whole image, a particular image detail or particular markings (implanted patient markers) in the images are optimally aligned. The images can be shifted either automatically by the system (for example, image fusion) or manually by the operator. Once shifted, the x-ray images and the DRRs are aligned as shown in the screen shot in FIG. 8. The 3D correlation of the 2D image pairs is taken into account.

Calculating the Patient Shift Necessary for Correct Placement

If the x-ray images have been shifted, the actual 3D patient positioning error is ascertained from this and therefore in turn the necessary correcting shift for the patient calculated. The screen shot in FIG. 9 shows the necessary correcting shift being indicated.

Correcting the Placement Error

Lastly, the patient is automatically (but “with computer monitoring”) moved to the correct treatment position.

Verifying the correct patient placement (optional)

In principle, the operator can then manually launch a second cycle of the entire sequence, for verification purposes. The system should then identify no further positioning errors within the framework of the system precision (approx. ±1 mm). This can also be performed such that in the verification procedure, no numerical values but rather only the superimposed images are indicated.

Monitoring the Patient During Treatment

During treatment, the system monitors the patient's position with the aid of the infrared body markers alone. In principle, verification can be repeated (periodically) with the aid of the x-ray system.

DE 10231630.9 filed Jul. 12, 2002 and the European application filed Jun. 18, 2003 and which claims priority of said German application, are both hereby incorporated herein in their entireties.

1. A patient positioning system for radiosurgery and/or radiotherapy, in particular with the aid of a LINAC and based on stereoscopic x-ray images, wherein the radiation path for producing the x-ray images runs from the x-ray sources to the image recorders, passes through the target area of the linear accelerator, and wherein the LINAC and the x-ray sources and the image recorders are arranged such that the x-ray detectors are not exposed to the radiation of the LINAC.

2. The patient positioning system as set forth in claim 1, characterised by one or more of the following features:
positioning is based on comparing x-ray images shot using the system and DRRs;
two kV x-ray sources are permanently let into the floor, left and right of the LINAC gantry;
two x-ray image recorders, for example a-Si detectors or conventional x-ray image intensifiers, are fixed to the ceiling in front of the gantry, to the left and right above the patient;
any possibility of collision between the LINAC and the x-ray apparatus is ruled out, in particular the gantry and the table can be rotated freely;
the radiation is to be administered in a number of doses/radiotherapy sessions until the target is necrotic, in particular in two, three, four, five or more sessions;
the x-ray images are taken sequentially, spaced out in time, in particular using one generator for both x-ray tubes, which is toggled.

3. A calibration system for a patient positioning system in accordance with claim 1, characterised by one or more of the following features:
for calibrating, a “phantom” is used which is provided with x-ray-visible markings which are clearly visible in the x-ray image;
said phantom is additionally fitted with other markers which are detected by an independent 3D measuring system, for example an IR tracking system;
the x-ray markers and the additional markers have a known, spatially fixed relationship to each other;
the spatial position of the individual x-ray markers is calculated using the position of the additional markers in relation to a fixed point fixed in space, in particular in relation to the linear accelerator, detected by the 3D measuring system;
the spatial position (X, Y, Z+ angle) of the x-ray sources and the x-ray image recorders relative to a fixed point fixed in space, in particular in relation to the linear accelerator, is calculated from the spatial position of the individual x-ray markers and their projections onto the x-ray image recorders;
the x-ray system is calibrated, independent of the calibration/referencing of the LINAC system.

4. An image recorder and/or detector arranging device to be used with a patient positioning system in accordance with claim 1, characterised by one or more of the following features:
the image recorder can be moved along the beam direction of the respective x-ray source;
the image recorder is moved manually or using motors;
for taking x-ray images, the image recorder is situated in a position near the patient;
when it is not required, the image recorder is situated in a position away from the patient;
in the position away from the patient, the image recorders do not obstruct the rotation of the gantry and the table.

5. A collision protection means to be used with a patient positioning system in accordance with claim 1 or an image recorder and/or detector arranging device in accordance with claim 4, characterised by one or more of the following features:
the image recorders are equipped with a collision protection system, for example a contact sensor, ultrasound telemeter, laser scanning;
the collision protection system enables the image recorders to be moved automatically.
